(19) United States
(12) Patent Application Publication
(10) Pub. No.: US 2024/0390551 A1
(43) Pub. Date: Nov. 28, 2024

(54) FILLER COMPOSITION FOR REDUCING SKIN WRINKLES COMPRISING STEM CELL-DERIVED EXOSOMES, HYALURONIC ACID, AND BDDE AND METHOD FOR PREPARING SAME

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Dong Gil YOU, Suwon-si (KR); Jae Yoon AN, Seoul (KR)

(73) Assignee: RESEARCH & BUSINESS FOUNDATION SUNGKYUNKWAN UNIVERSITY, Suwon-si (KR)

(21) Appl. No.: 18/694,586

(22) PCT Filed: Jan. 18, 2022
(86) PCT No.: PCT/KR2022/000894

§ 371 Date: Mar. 22, 2024

(30) Foreign Application Priority Data
Jan. 20, 2021 (KR) 10-2021-0008276
Dec. 29, 2021 (KR) 10-2021-0190819

(51) Int. Cl. A61L 27/36; A61L 27/20; A61L 27/48; A61L 27/52; A61L 27/54

(52) U.S. Cl. CPC A61L 27/3604; A61L 27/20; A61L 27/3687; A61L 27/48; A61L 27/52; A61L 27/54; A61L 2300/626; A61L 2430/34

(57) ABSTRACT

The present invention relates to: a filler composition for reducing skin wrinkles, comprising stem cell-derived exosomes, hyaluronic acid, and BDDE; and a method for preparing same. The filler composition for reducing skin wrinkles according to the present invention not only overcome limitations of low in-vivo engraftment rate and survival rate of cells, which are the biggest problems of treatment using stem cells, but also suppresses side effects caused by tumorigenesis of stem cells and the like and exhibits an excellent collagen production effect, and it has been confirmed that the filler composition increases the proliferation of fibroblasts and collagen production by activating anti-inflammatory macrophages. Thus, the filler composition is expected to be effectively used in treatments for anti-aging, filler injections for cosmetic purposes, or the like.

### TECHNICAL FIELD

The present invention relates to a filler composition for reducing skin wrinkles containing stem cell-derived exosomes, hyaluronic acid, and BDDE, and a method for preparing the same.

This application claims priorities based on Korean Patent Application No. 10-2021-0008276, filed on Jan. 20, 2021, and Korean Patent Application No. 10-2021-0190819, filed on Dec. 29, 2021, and all the contents disclosed in the specifications and drawings of those applications are incorporated in this application.

### BACKGROUND ART

The skin is divided into three layers: the epidermis, which is an outer layer, the dermis, which is an inner layer, and subcutaneous tissue. The skin has a structure in which a basement membrane exists between the epidermis and the dermis. The skin is a tissue that plays an important role in maintaining body water and body temperature while preventing the entry of bacteria from the outside.

The dermis is the largest portion of the skin, and is filled with a macromolecular network structure called “extracellular matrix”. This extracellular matrix is produced by fibroblasts in the dermis, and is composed of polysaccharides called acidic mucopolysaccharides, such as hyaluronic acid (HA) or dermatan sulfate, and fibrous proteins such as collagen, and elastin. The extracellular matrix is directly involved in the elasticity, tightness, moistness, metabolism, etc. of the skin, and the main causes of skin aging are a decrease in skin elasticity and an increase in wrinkles due to the degeneration of collagen, elastin, and glycosaminoglycans and mucopolysaccharides. If there is a lack of collagen in the skin layer, the thickness of the skin layer becomes thinner, which reduces elasticity and causes wrinkles. Generally, as aging progresses, the number of anti-inflammatory macrophages present in the skin layer tends to decrease and the number of inflammatory macrophages tends to increase.

Currently, research and development on fillers is being actively conducted for the purpose of reducing skin wrinkles, and representative fillers are hyaluronic acid-based fillers.

Hyaluronic acid (HA) has many functions, including the maintenance of water in intercellular spaces, the maintenance of cells based on forming a jelly-like matrix in tissue, the maintenance of tissue lubricity and flexibility, resistance to external forces such as mechanical stress, and prevention of cellular infection.

The conventional fillers based on hyaluronic acid as described above have little toxicity in vivo and have the advantage of creating an enlargement effect by directly increasing the volume immediately after injection, but have short-term effects and have limitations in producing additional collagen.

Recently, a variety of anti-aging therapeutic methods using stem cells have been introduced, but it is difficult to regulate the differentiation of the cells themselves, and side effects may occur due to in vivo immune response in the case of allogeneic transplantation. In addition, the in vivo viability of the cells is very low, which may lead to limitations in direct clinical application.

Meanwhile, exosomes are vesicles having the same membrane structure as the cell membrane, and are known to play a role in delivering membrane components, proteins, RNA, etc. to other cells and tissues. In particular, it is known that exosomes secreted from stem cells contain not only receptors and proteins, but also nuclear components, and thus play a role in intercellular communication, and they also contain various growth factors and cytokines secreted by stem cells, and thus regulate behaviors such as cell adhesion, growth and differentiation. In addition, since impurities such as cell debris, antibiotics, serum, etc. in cell cultures are removed in the isolation process, exosomes can be safely used while having the same effect as that of cell cultures.

Accordingly, the present inventors extracted exosomes from stem cells, and added the extracted exosomes to an existing hyaluronic acid-based filler composition without changing the physical properties of the filler, to develop a filler composition having excellent wrinkle-reducing effects while having minimized side effects.

### DISCLOSURE

### Technical Problem

The present inventors have conducted studies to develop a filler composition for reducing wrinkles, which has excellent effects while overcoming problems that may occur in conventional hyaluronic acid-based filler compositions and anti-aging therapy using stem cells, and as a result, have found that, when stem cell-derived exosomes are added to hyaluronic acid, they exhibit an excellent collagen production effect while minimizing side effects. Furthermore, the present inventors have found the optimal addition ratio of BDDE to hyaluronic acid by evaluating the collagen production effect at each content of BDDE added as a crosslinking agent to hyaluronic acid. In addition, the present inventors have found that a filler composition containing stem cell-derived exosomes increases fibroblast proliferation and collagen production by activating anti-inflammatory macrophages. Based on these findings, the present invention has been completed.

Accordingly, an object of the present invention is to provide a filler composition for reducing skin wrinkles, containing stem cell-derived exosomes, hyaluronic acid, and 1,4-butanediol diglycidyl ether (BDDE) as active ingredients, and a method for preparing the same.

However, objects to be achieved by the present invention are not limited to the objects mentioned above, and other objects not mentioned above will be clearly understood by those skilled in the art to which the present invention belongs from the following description below.

### Technical Solution

To achieve the above object, the present invention provides a filler composition for reducing skin wrinkles, containing exosomes derived from stem cells, hyaluronic acid, and 1,4-butanediol diglycidyl ether (BDDE) as active ingredients.

The present invention also provides a method for preparing a filler composition for reducing skin wrinkles, comprising steps of:
(a) extracting exosomes from stem cells;
(b) crosslinking hyaluronic acid by adding BDDE thereto;
(c) dialyzing the crosslinked hyaluronic acid using a dialysis membrane; and
(d) mixing the cross-linked hyaluronic acid after the dialyzing with the exosomes.

In one embodiment of the present invention, the stem cells may be human adipose stem cells, without being limited thereto.

In another embodiment of the present invention, the hyaluronic acid may be crosslinked with BDDE to form a hydrogel, without being limited thereto.

In another embodiment of the present invention, the dry weight ratio of hyaluronic acid: BDDE may be 1:0.001 to 0.05, without being limited thereto.

In another embodiment of the present invention, the filler composition is able to activate anti-inflammatory macrophages, without being limited thereto.

In another embodiment of the present invention, the filler composition is able to increase the expression of CD301b, an anti-inflammatory marker, in macrophages, without being limited thereto.

In another embodiment of the present invention, the filler composition is able to increase fibroblast proliferation and collagen production by activating anti-inflammatory macrophages, without being limited thereto.

In another embodiment of the present invention, the crosslinking in step (b) may be performed at a temperature of 20° C. to 60° C. for 12 to 36 hours, without being limited thereto.

In another embodiment of the present invention, the dialyzing in step (c) may be performed for 36 to 60 hours, without being limited thereto.

The present invention also provides the use of a filler composition, containing exosomes derived from stem cells, hyaluronic acid, and 1,4-butanediol diglycidyl ether (BDDE) as active ingredients, for reducing skin wrinkles.

The present invention provides also the use of exosomes derived from stem cells, hyaluronic acid, and 1,4-butanediol diglycidyl ether (BDDE) for preparing a filler for reducing skin wrinkles.

The present invention also provides a method for reducing skin wrinkles, comprising a step of administering to a subject in need thereof a filler composition containing exosomes derived from stem cells, hyaluronic acid, and 1,4-butanediol diglycidyl ether (BDDE) as active ingredients.

### Advantageous Effects

The filler composition for reducing skin wrinkles according to the present invention contains human adipose stem cell-derived exosomes, hyaluronic acid, and BDDE. The filler composition not only overcomes the limitations of the low engraftment rate and viability of cells, which are the biggest problems occurring in therapy using stem cells, but also exhibits excellent collagen production effects while suppressing side effects such as tumorigenesis of stem cells. It has been found that the filler composition increases fibroblast proliferation and collagen production by activating anti-inflammatory macrophages. In addition, the exosomes may be contained in a hyaluronic acid filler composition without changing the physical properties of the existing hyaluronic acid filler, and even if the hyaluronic acid filler composition is decomposed, wrinkles in the injected area may be fundamentally reduced by collagen production.

### BRIEF DESCRIPTION OF DRAWINGS

FIG. 1 schematically shows the collagen production action of a hydrogel filler containing human adipose stem cell-derived exosomes and hyaluronic acid crosslinked with BDDE according to one embodiment of the present invention.

FIG. 2 shows the chemical structure of crosslinked hyaluronic acid according to one embodiment of the present invention.

FIG. 3a shows the results of morphological analysis of hyaluronic acid fillers containing or not containing human adipose stem cell-derived exosomes according to one embodiment of the present invention.

FIG. 3b shows confocal fluorescence microscope images of hyaluronic acid fillers containing or not containing human adipose stem cell-derived exosomes according to one embodiment of the present invention.

FIG. 3c shows scanning electron microscope images of hyaluronic acid fillers containing or not containing human adipose stem cell-derived exosomes according to one embodiment of the present invention.

FIG. 4a shows the results of analyzing the rheological properties of hyaluronic acid fillers containing or not containing human adipose stem cell-derived exosomes according to one embodiment of the present invention.

FIG. 4b shows the results of analyzing the injection forces of hyaluronic acid fillers containing or not containing human adipose stem cell-derived exosomes according to one embodiment of the present invention.

FIG. 5a shows the results of evaluating the biodistribution behavior of exosomes depending on the content of BDDE in hyaluronic acid fillers containing human adipose stem cell-derived exosomes according to one embodiment of the present invention.

FIG. 5b shows the results of evaluating the biodistribution behavior of exosomes 2 days after injection of hyaluronic acid fillers containing human adipose stem cell-derived exosomes according to one embodiment of the present invention.

FIG. 5c shows the results of evaluating the long-term biodistribution behavior of exosomes in a hyaluronic acid filler containing human adipose stem cell-derived exosomes, which has an optimal BDDE content, according to one embodiment of the present invention.

FIG. 6a shows the results of staining mouse skin layer tissue 24 weeks after injection of hyaluronic acid fillers containing human adipose stem cell-derived exosomes according to one embodiment of the present invention.

FIG. 6b shows the results of quantitatively analyzing collagen in the dermal layer 24 weeks after injection of hyaluronic acid fillers containing human adipose stem cell-derived exosomes according to one embodiment of the present invention.

FIG. 6c shows the results of quantitatively analyzing the thickness of the dermal layer in which collagen is distributed, 24 weeks after injection of hyaluronic acid fillers containing human adipose stem cell-derived exosomes according to one embodiment of the present invention.

FIG. 7 shows the results of analyzing the production of collagen I and III in skin tissue after injection of hyaluronic acid fillers containing human adipose stem cell-derived exosomes according to one embodiment of the present invention (scale bar=50 μm).

FIG. 8a shows the results of evaluating the expression level of the anti-inflammatory marker CD301b in macrophages of various phenotypes, present in the skin layer and activated by human adipose stem cell-derived exosomes according to one embodiment of the present invention (scale bar=50 μm).

FIG. 8b shows the results of quantitatively analyzing CD301b expressed on the surface of macrophages treated with human adipose stem cell-derived exosomes according to one embodiment of the present invention.

FIG. 9a shows the results of quantitatively analyzing the number of proliferated fibroblasts by cytotoxicity assay after treatment with human adipose stem cell-derived exosomes according to one embodiment of the present invention.

FIG. 9b shows the results of quantitatively analyzing the proliferation rate of fibroblasts by activated macrophages after co-culture of fibroblasts and the macrophages activated by treatment with human adipose stem cell-derived exosomes according to one embodiment of the present invention.

FIG. 10a shows the results of quantitatively analyzing collagen synthesized by fibroblasts after treatment with human adipose stem cell-derived exosomes according to one embodiment of the present invention.

FIG. 10b shows the results of quantitatively analyzing the collagen synthesis effect of fibroblasts induced by activated macrophages after co-culture of fibroblasts and the macrophages activated by treatment with human adipose stem cell-derived exosomes according to one embodiment of the present invention.

FIG. 11a shows the results of analyzing the expression of CD301b, an anti-inflammatory macrophage marker, in skin tissue after injecting an exosome-containing hyaluronic acid filler according to one embodiment of the present invention into a mouse model (scale bar=100 μm).

FIG. 11b shows the results of quantitatively analyzing the expression level of CD301b in skin tissue after injecting an exosome-containing hyaluronic acid filler according to one embodiment of the present invention into a mouse model.

FIG. 12a shows the results of analyzing the proliferation of fibroblasts in skin tissue after injecting an exosome-containing hyaluronic acid filler according to one embodiment of the present invention into a mouse model (scale bar=50 μm).

FIG. 12b shows the results of quantitatively analyzing the number of fibroblasts in skin tissue after injecting an exosome-containing hyaluronic acid filler according to one embodiment of the present invention into a mouse model.

### BEST MODE

The present inventors have developed a filler composition for reducing skin wrinkles, which contains human adipose exosomes derived from stem cells, hyaluronic acid, and BDDE and has an excellent collagen production effect while having minimized side effects. Furthermore, the present inventors have found the optimal addition ratio of BDDE to hyaluronic acid by evaluating the collagen production effect at each content of BDDE used as a crosslinking agent.

In examples of the present invention, exosomes were extracted from human adipose stem cells (see Example 1), hyaluronic acid was crosslinked by adding BDDE, and then the exosomes were mixed with the crosslinked hyaluronic acid, thereby preparing a filler (see Example 2 and 3).

In another example of the present invention, as a result of analyzing the properties of hyaluronic acid fillers containing or not containing human adipose stem cell-derived exosomes, it was found that the contained exosomes did not affect changes in the transparency and color of the hyaluronic acid filler and were evenly distributed in the hyaluronic acid filler. In addition, it was found that the contained exosomes did not affect the storage modulus, loss modulus, and injection force of the hyaluronic acid filler (see Example 4).

In one experimental example of the present invention, as a result of evaluating the biodistribution behavior of exosomes depending on the content of BDDE in a hyaluronic acid filler containing human adipose stem cell-derived exosomes, it confirmed found that the exosomes present in the exosome-containing hyaluronic acid filler having a BDDE content of 108.46 mg remained in the skin layer for the longest period of time, indicating that the optimal content of BDDE is 108.46 mg (see Experimental Example 1).

In another experimental example of the present invention, as a result of evaluating the anti-aging effect of a hyaluronic acid filler containing human adipose stem cell-derived exosomes in a mouse model, it was confirmed that the hyaluronic acid filler containing human adipose stem cell-derived exosomes according to the present invention increased collagen production and the thickness of the dermal layer in an exosome concentration-dependent manner (see Experimental Example 2).

In another experimental example of the present invention, as a result of evaluating the collagen production effect of human adipose stem cell-derived exosomes by activation of anti-inflammatory macrophages, it could be seen that the human adipose stem cell-derived exosomes had significant effect on CD301b expression in macrophages involved in anti-inflammatory responses, and increased the number of fibroblasts by activating anti-inflammatory macrophages in the skin layer, thereby increasing collagen synthesis (see Experimental Example 3).

In another experimental example of the present invention, as a result of evaluating the effect of injection of the exosome-containing hyaluronic acid filler on the improvement of the micro-environment in the skin layer, it could be seen that, when the exosome-containing hyaluronic acid filler according to the present invention was injected into an animal model, the expression of CD301b, an anti-inflammatory macrophage marker, significantly increased and the number of fibroblasts increased, compared to when the exosomes were injected alone (see Experimental Example 4).

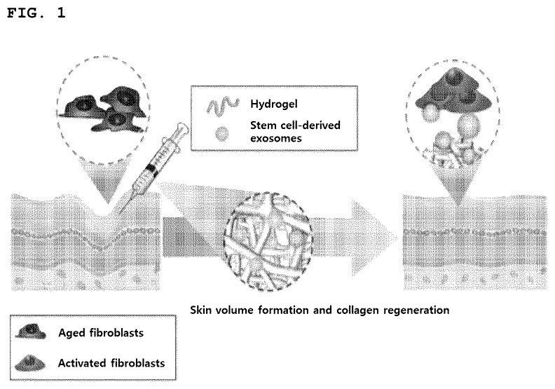

FIG. 1 schematically shows the collagen production action of a hydrogel filler containing human adipose stem cell-derived exosomes and hyaluronic acid crosslinked with BDDE according to the present invention.

Accordingly, the present invention provides a filler composition for reducing skin wrinkles, containing exosomes derived from stem cells, hyaluronic acid, and 1,4-butanediol diglycidyl ether (BDDE) as active ingredients.

The present invention also provides the use of a filler composition, containing exosomes derived from stem cells, hyaluronic acid, and 1,4-butanediol diglycidyl ether (BDDE) as active ingredients, for reducing skin wrinkles.

The present invention provides also the use of exosomes derived from stem cells, hyaluronic acid, and 1,4-butanediol diglycidyl ether (BDDE) for preparing a filler for reducing skin wrinkles.

The present invention also provides a method for reducing skin wrinkles, comprising a step of administering to a subject in need thereof a filler composition containing exosomes derived from stem cells, hyaluronic acid, and 1,4-butanediol diglycidyl ether (BDDE) as active ingredients.

In the present invention, the term “stem cell” refers, in a broad sense, to undifferentiated cells having the ability to differentiate into various types of tissue cells, that is, stemness, and includes not only pluripotent stem cells, which can differentiate into all types of cells that make up living organisms, such as nerves, blood, and cartilage, but also multipotent and unipotent stem cells. These stem cells are broadly divided into embryonic stem cells, which can be produced using embryos, adult stem cells, gametes, and cancer stem cells. Embryonic stem cells refer to the cell mass stage prior to forming specific organs within 14 days after fertilization, and recently, embryonic stem cells have also been produced from normal cells through dedifferentiation. Thus, stem cells are not limited as long as they are cells capable of differentiating into all types of cells and tissues constituting the body. Adult stem cells are extracted from umbilical cord blood, bone marrow, blood, and the like, and refer to primitive cells immediately before differentiation into cells of specific organs such as bone, the liver, blood, and the like. Gametes are cells that pass down genetic information to the next generation through reproduction, and include human sperms and eggs, without being limited thereto.

In addition, stem cells are cells that can self-replicate in the process of clustering cells by forming clones, maintain new single stem cells within the cluster, and are capable of differentiating into one or more specialized cell types.

In the present invention, the term “adipose-derived stem cells (ASCs)” refers to stem cells extracted from adipose tissue, among adult stem cells derived from various sources such as bone, muscle, adipose, and umbilical cord blood. Multipotent adipose-derived stem cells (ASCs) are capable of differentiating into most mesenchymal cells, such as adipocytes, osteoblasts, chondroblasts, and myofibroblasts.

In the present invention, the adipose-derived stem cells may be human adipose-derived stem cells, without being limited thereto.

In the present invention, the term “exosomes” refers to small membrane vesicles secreted from various cells. In a study using an electron microscope, it was observed that exosomes originate from specific intracellular compartments called multivesicular bodies (MVBs) and are released and secreted out of the cell, rather than directly detach from the plasma membrane. That is, when fusion of the multivesicular bodies and the plasma membrane occurs, vesicles are released into the extracellular environment, which are called the extracellular vesicles. Although it is not clear by what molecular mechanism these exosomes are made, it is known that not only red blood cells, but also various types of immune cells, including B-lymphocytes, T-lymphocytes, dendritic cells, platelets, and macrophages, as well as tumor cells and stem cells produce and secrete exosomes when they are alive. The exosomes include those secreted naturally or artificially.

In the present invention, the exosomes may have a diameter of 10 nm to 500 nm, 10 nm to 400 nm, 10 nm to 300 nm, 10 nm to 250 nm, 10 nm to 200 nm, 10 nm to 150 nm, 50 nm to 500 nm, 50 nm to 400 nm, 50 nm to 300 nm, 50 nm to 200 nm, 50 nm to 150 nm, or 80 nm to 130 nm, without being limited thereto.

In the present invention, the exosomes may be injected at 1×10⁶ to 1×10⁹, 1×10⁷ to 1×10⁹, 1×10⁶ to 1×10⁸, 5×10⁶ to 1×10⁹, 5×10⁶ to 1×10⁸, 5×10⁷ to 1×10⁹, 1×10⁷ to 1×10⁸, or 1×10⁸ exosomes per filler-injected subject, without being limited thereto.

In the present invention, “hyaluronic acid (HA)” is one of glycosaminoglycans and consists of N-acetyl glucosamine and glucuronic acid. It is known that hyaluronic acid is present in the extracellular matrix, is involved in tissue water retention, storage and diffusion of cell growth factors and nutrients, and is synthesized by keratinocytes and fibroblasts. A decrease in hyaluronic acid in the skin is known to be the cause of decreased skin elasticity and increased wrinkles. Accordingly, maintaining the hyaluronic acid content in the skin plays an important role not only in moisturizing and maintaining skin elasticity, but also in anti-aging skin care such as wrinkle reduction.

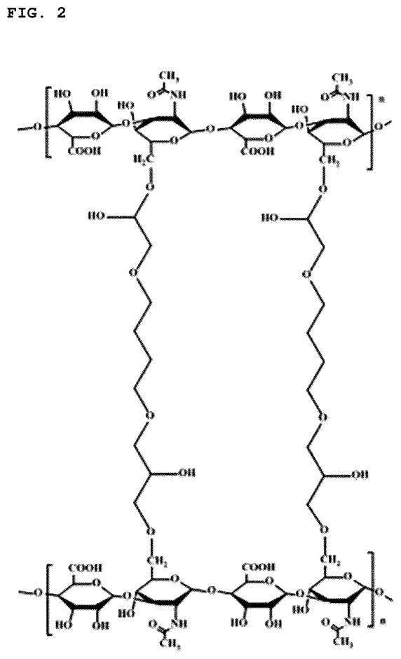

In the present invention, “1, 4-butanediol diglycidyl ether (BDDE)” may function as a crosslinking agent for crosslinking hyaluronic acid. FIG. 2 shows the chemical structure of hyaluronic acid crosslinked with BDDE. Hyaluronic acid may be crosslinked with BDDE to form a hydrogel.

In the present invention, the term “crosslinking” refers to methods of effectively rendering normally water-soluble materials substantially water-insoluble but swellable. Such methods include, for example, physical entanglement, crystalline domains, covalent bonds, ionic complexes and associations, hydrophilic associations such as hydrogen bonding, hydrophobic associations, or Van der Waals forces.

In the present invention, the dry weight ratio of hyaluronic acid: BDDE that are contained in the filler composition may be 1:0.001 to 0.05, 1:0.001 to 0.04, 1:0.001 to 0.03, 1:0.002 to 0.05, 1:0.002 to 0.04, 1:0.002 to 0.03, 1:0.01 to 0.05, 1:0.01 to 0.04, 1:0.01 to 0.03, 1:0.02 to 0.05, 1:0.02 to 0.04, or 1:0.02 to 0.03, without being limited thereto.

In the present invention, the filler composition is able to activate anti-inflammatory macrophages, without being limited thereto. In this case, the filler composition is able to increase the expression of CD301b, an anti-inflammatory marker, in macrophages, and is able to increase fibroblast proliferation and collagen production by activation of anti-inflammatory macrophages, without being limited thereto.

Accordingly, in another aspect of the present invention, the present invention may provide a filler composition for activating macrophages in the skin layer, the filler composition containing stem cell-derived exosomes, hyaluronic acid, and 1,4-butanediol diglycidyl ether (BDDE) as active ingredients.

In the present invention, “macrophages” refers to immune cells that originate from bone marrow cells and are responsible for the main functions of representative innate immunity. Initially, macrophages leave the bone marrow through the bloodstream in the form of immature monocytes. In the process of recognizing the by-products or pathogens derived from infected cells, monocytes increase their activity and differentiate into mature macrophages. Macrophages play an important function in maintaining tissue homeostasis by removing invading pathogens and inducing adaptive immunity. Macrophages that activate Th1 T lymphocytes provide an inflammatory response and are designated as M1 macrophages. M1 macrophages (also referred to as “killer macrophages”) inhibit cell proliferation, cause tissue damage, and are aggressive against bacteria. Macrophages that activate Th2 T lymphocytes provide an anti-inflammatory response and are designated as M2 macrophages. M2 macrophages (also referred to as “repair macrophages”) are important in cellular homeostasis and inflammatory responses, promote cell proliferation and tissue repair, and are anti-inflammatory. In the present invention, general macrophages excluding the M1 and M2 macrophages are denoted M0.

In the present invention, “activation of anti-inflammatory macrophages” refers to a state in which anti-inflammatory macrophages are sufficiently stimulated to express anti-inflammatory markers such as CD301b and increase fibroblast proliferation and resulting collagen production.

In the present invention, the term “skin wrinkles” refers to fine lines caused by aging of the skin. Skin wrinkles can be caused by genetic factors, reduction in collagen and elastin present in the skin dermis, external environment, etc. In the present invention, “reducing skin wrinkles” means suppressing or inhibiting the formation of wrinkles on the skin or alleviating already formed wrinkles.

In the present invention, the term “filler” refers broadly to a material or composition designed to add volume to deficient areas of soft tissues that connect, support, or surround other structures and organs of the body, such as muscles, tendons, fibrous tissue, fat, blood vessels, nerves, and synovial tissue (tissue around joints). Preferably, the term “filler” refers to a material that can fill the skin by being injected or inserted into the skin to reduce skin wrinkles.

In the present invention, the filler composition containing stem cell-derived exosomes, hyaluronic acid, and BDDE may be further processed by mixing with, for example, water or a saline solution to form an injectable or topical substance such as a solution, oil, lotion, gel, ointment, cream, slurry, salve, or paste. According to one embodiment of the present invention, the filler composition may be injectable, without being limited thereto.

According to one embodiment of the present invention, the formulation of the injectable filler composition may be a gel. The gel generally refers to a material having fluidity at room temperature between that of a liquid and solid. Specifically, the gel may be a hydrogel capable of absorbing water. According to one embodiment of the present invention, the present invention may provide an injectable filler composition in the form of a gel, which has an appropriate viscosity by containing human adipose stem cell-derived exosomes and hyaluronic acid crosslinked with BDDE.

In the present invention, the injectable filler composition may contain a solvent such as distilled water for injection, a 0.9% sodium chloride injection, a Ringer's solution, a dextrose injection, a dextrose+sodium chloride injection, PEG, lactated Ringer's solution, ethanol, propylene glycol, non-volatile oil-sesame oil, cottonseed oil, peanut oil, soybean oil, corn oil, ethyl oleate, isopropyl myristate, or benzene benzoate; a solubilizer such as sodium benzoate, sodium salicylate, sodium acetate, urea, urethane, monoethylacetamide, butazolidine, propylene glycol, Tween, nicotinic acid amide, hexamine, or dimethylacetamide; a buffer such as a weak acid and a salt thereof (acetic acid and sodium acetate), a weak base and a salt thereof (ammonia and ammonium acetate), an organic compound, a protein, albumin, peptone, or a gum; an isotonic agent such as sodium chloride; a stabilizer such as sodium bisulfite (NaHSO₃) carbon dioxide gas, sodium metabisulfite (Na₂S₂O₅), sodium sulfite (Na₂SO₃), nitrogen gas (N₂), or ethylenediamine tetraacetic acid; an antioxidant such as 0.1% sodium bisulfide, sodium formaldehyde sulfoxylate, thiourea, disodium ethylenediaminetetraacetic acid, or acetone sodium bisulfite; a pain-relieving agent such as benzyl alcohol, chlorobutanol, procaine hydrochloride, glucose, or calcium gluconate; or a suspending agent such as CMC Na, sodium alginate, Tween 80, or aluminum monostearate.

In the present invention, the filler composition may be may be administered for the treatment or amelioration of, for example, wrinkles or lines of the skin (e.g., facial lines and facial wrinkles), glabellar lines, nasolabial folds, chin folds, marionette lines, buccal commissures, fine wrinkles around the eyes, cutaneous depressions, scars, temples, subdermal support of the brows, malar and buccal fat pads, tear troughs, nose, lips, cheeks, perioral region, infraorbital region, facial asymmetries, jawlines, and chin, and may be administered by any means known to those skilled in the art, including, without limitation, syringe with needle, a pistol (for example, a hydropneumatic-compression pistol), catheter, topically, or by direct surgical implantation. In this case, the needle may be combined with a syringe, catheter, and/or a pistol.

In the present invention, the filler composition may be administered, for example, to a skin area such as a dermal area (intradermal injection) or a subcutaneous area, without being limited thereto.

In the present invention, the filler composition may be administered once or multiple times, and the administration duration and dosage thereof may typically be determined based on the cosmetic and/or clinical effect desired by the individual and/or physician and the body part or region being treated, without being particularly limited thereto.

In the present invention, the term “administration” means providing a given composition of the present invention to an individual by any appropriate method.

In the present invention, the term “individual” refers to a subject in need of skin wrinkle reduction and to which the composition of the present invention may be administered. More specifically, it refers to humans or non-human primates, mammals such as mice, dogs, cats, horses, and cows, etc.

The present invention also provides a method for preparing a filler composition for reducing skin wrinkles, comprising steps of:
(a) extracting exosomes from stem cells;
(b) crosslinking hyaluronic acid by adding BDDE thereto;
(c) dialyzing the crosslinked hyaluronic acid using a dialysis membrane; and
(d) mixing the cross-linked hyaluronic acid after the dialyzing with the exosomes.

In the present invention, step (a) may comprises steps of: culturing human adipose-derived stem cells in a normal culture medium, and replacing the medium with a serum-free, antibiotic-free, phenol red-free medium 12 to 36 hours, 12 to 30 hours, 18 to 36 hours, 18 to 30 hours, or 24 hours extracting exosomes, followed by culturing for 12 to 36 hours, 12 to 30 hours, 18 to 36 hours, 18 to 30 hours, or 24 hours;

recovering the cell culture supernatant, and centrifuging the recovered cell culture supernatant at 1,000×g to 3,000×g, 1,500×g to 3,000×g, 1,000×g to 2,500×g, 1,500×g to 2,500×g, or 2,000×g for 1 min to 10 min, 1 min to 7 min, 3 min to 10 min, 3 min to 7 min, or 4 min to 5 min, followed by centrifugation at 5,000×g to 15,000×g, 5,000×g to 12,000×g, 7,000×g to 15,000×g, 7,000×g to 12,000×g, or 10,000×g for 1 min to 50 min, 1 min to 40 min, 3 min to 50 min, 3 min to 40 min, or 4 min to 30 min, thereby removing cellular debris and waste;
centrifuging the recovered cell culture supernatant at 1,000×g to 5,000×g, 1,000×g to 4,000×g, 2,000×g to 5,000×g, 2,000×g to 4,000×g, or 3,000×g and 0° C. to 10° C., 2° C. to 8° C., 2° C. to 6° C., or 4° C. for 10 min to 30 min, 15 min to 25 min, or 20 min, followed by filtration through a 0.01 μm to 0.5 μm, 0.05 μm to 0.4 μm, 0.1 μm to 0.3 μm, or 0.22 μm filter, thereby removing cellular debris and waste; and
filtering the filtered cell culture supernatant using a tangential flow filtration system with a 100 kDa to 500 kDa, 100 kDa to 400 kDa, 200 kDa to 500 kDa, 200 kDa to 400 kDa, or 300 kDa filter, thereby isolating and purifying exosomes.

In the present invention, step (b) may be a step of dissolving hyaluronic acid: BDDE at a dry weight ratio of 1:0.001 to 0.05, 1:0.001 to 0.04, 1:0.001 to 0.03, 1:0.002 to 0.05, 1:0.002 to 0.04, 1:0.002 to 0.03, 1:0.01 to 0.05, 1:0.01 to 0.04, 1:0.01 to 0.03, 1:0.02 to 0.05, 1:0.02 to 0.04, or 1:0.02 to 0.03 in a sodium hydroxide solution, followed by a crosslinking reaction.

In this case, in step (b), the hyaluronic acid may have a molecular weight of 500 kDa to 2,000 kDa, 500 kDa to 1,700 kDa, 500 kDa to 1,500 kDa, 500 kDa to 1,200 kDa, 700 kDa to 2,000 kDa, 700 kDa to 1,700 kDa, 700 kDa to 1,500 kDa, 700 kDa to 1,200 kDa, 800 kDa to 2,000 kDa, 800 kDa to 1,700 kDa, 800 kDa to 1,500 kDa, 800 kDa to 1,100 kDa, or 1,000 kDa, and the crosslinking reaction may be performed at a temperature of 20° C. to 60° C., 20° C. to 50° C., 30° C. to 60° C., 30° C. to 50° C., or 40° C. for 12 hours to 36 hours, 12 hours to 30 hours, 18 hours to 36 hours, 18 hours to 30 hours, or 24 hours, without being limited thereto.

In the present invention, step (c) is a step of dialyzing the crosslinked hyaluronic acid with phosphate-buffered saline (PBS) using a dialysis membrane to purify and swell the crosslinked hyaluronic acid. Here, the dialyzing may be performed for 36 hours to 60 hours, 36 hours to 54 hours, 42 hours to 60 hours, 42 to 54 hours, or 48 hours, without being limited thereto.

In step (d) of the present invention, the exosomes may be mixed to a concentration of 1×10⁷ to 1×10¹⁰, 1×10⁸ to 1×10¹⁰, 1×10⁷ to 1×10⁹, 1×10⁸ to 1×10⁹, 5×10⁷ to 1×10¹⁰, 5×10⁷ to 1×10⁹, 5×10⁸ to 1×10⁹, or 1×10⁹ exosomes per ml of the sodium hydroxide solution in which hyaluronic acid and BDDE have been dissolved in step (b), without being limited thereto.

Hereinafter, preferred examples will be presented to help understand the present invention. However, the following examples are provided only for a better understanding of the present invention, and the scope of the present invention is not limited by the following examples.

### MODE FOR INVENTION

### Examples

### Example 1. Extraction of Human Adipose Stem Cell-Derived Exosomes

Human adipose stem cell-derived exosomes were extracted during the process of culturing human adipose stem cells.

Specifically, human adipose stem cells were cultured in a normal culture medium (Gibco, Cat #: 11995065), and 24 hours before exosome extraction, the medium was replaced with a serum-free, antibiotic-free, phenol red-free medium (Gibco, Cat #: 31053028), and the cells were cultured in the replaced medium for 24 hours, followed by recovery of the cell culture supernatant. The recovered cell culture supernatant was first centrifuged at 2,000×g for 4 to 5 minutes and then centrifuged at 10,000×g for 4 to 30 minutes to remove cellular debris and waste. Thereafter, the recovered cell culture supernatant was first centrifuged at 3,000×g and 4° C. for 20 minutes and then filtered through a 0.22 μm filter, thereby removing cellular debris and waste. Next, exosomes were isolated and purified from the recovered supernatant using a tangential flow filtration (TFF) system with a 300 kDa filter.

### Example 2. Optimization of Hyaluronic Acid Filler Synthesis Conditions Depending on Content of BDDE

Using a centrifugal mixer for high viscosity material, 5 g of hyaluronic acid (molecular weight: 1,000 KDa) and each content of BDDE (1,4-butanediol diglycidyl ether) were dissolved in 25 mL of a 0.1 N sodium hydroxide (NaOH) solution, and then subjected to a crosslinking reaction at a temperature of 40° C. for 24 hours (on a dry weight basis, level 1:0 mg, level 2:10.85 mg, level 3:54.23 mg, level 4:108.46 mg, level 5:162.69 mg, and level 6:216.92 mg). After completion of the reaction, the hydrogel product was dialyzed with 10 mM phosphate-buffered saline (PBS) using a dialysis membrane (molecular weight cutoff: 12 to 14 KDa) for 48 hours to purify and swell the hydrogel product to a final concentration (20 mg/mL).

### Example 3. Preparation of Hyaluronic Acid Filler Containing Human Adipose Stem Cell-Derived Exosomes

The hydrogel produced by dissolving 5 g of hyaluronic acid and 108.46 mg of BDDE (1,4-butanediol diglycidyl ether) in 25 mL of a 0.1 N sodium hydroxide (NaOH) solution, followed by a crosslinking solution, according to the method of Example 2, was mixed with the human adipose stem cell-derived exosome solution extracted in Example 1, thereby preparing a hyaluronic acid filler containing the exosomes. At this time, the human adipose stem cell-derived exosomes extracted in Example 1 were mixed to a concentration of 1×10⁹ exosomes per ml of the hydrogel solution obtained by dissolving hyaluronic acid and BDDE in the sodium hydroxide solution.

### Example 4. Characterization of Hyaluronic Acid Filler Containing or Not Containing Exosomes

### 4-1. Morphological Analysis and Image Observation

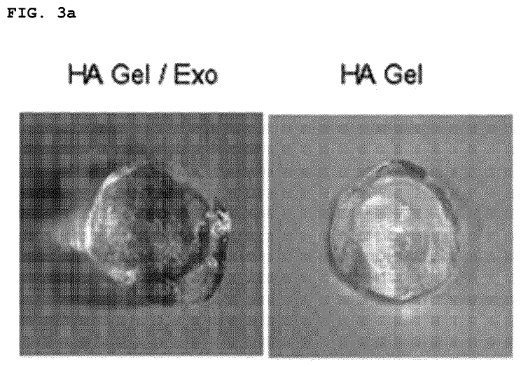

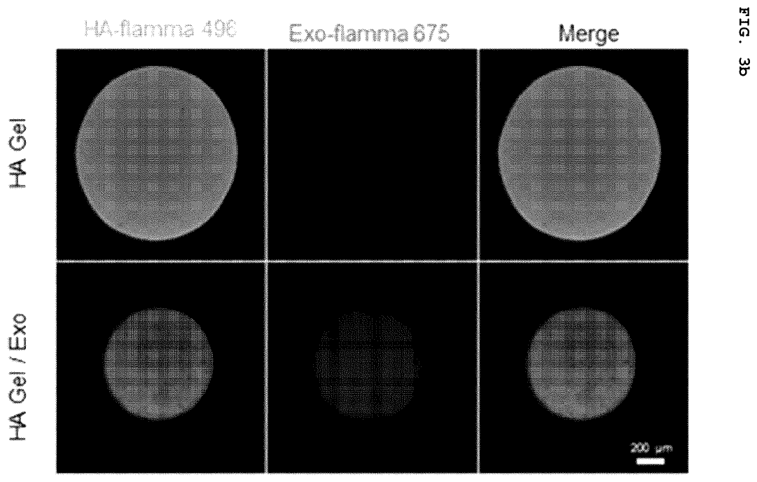

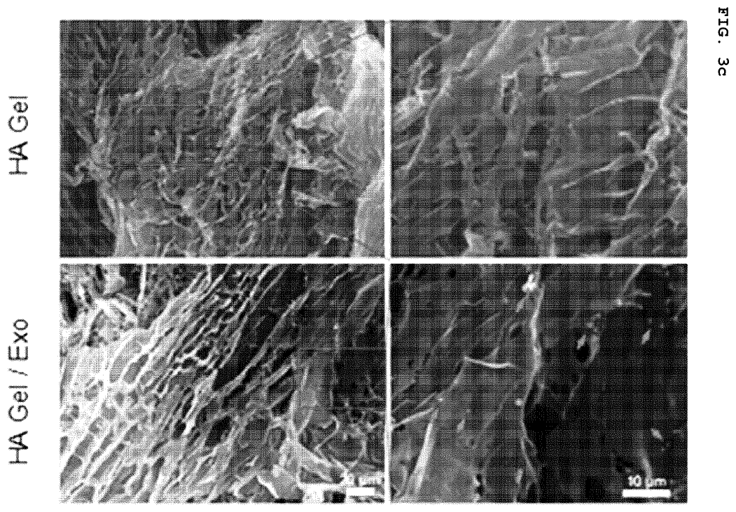

A hyaluronic acid filler containing exosomes and a hyaluronic acid filler not containing exosomes were prepared according to the method of Example 3, and each of the hyaluronic acid filler solutions was injected onto the bottom using a 1 ml syringe having a needle size of 31G. The results of comparing the morphologies of the injected hydrogels are shown in FIG. 3a.

FIG. 3a shows the results of morphological analysis of the hyaluronic acid fillers containing or not containing the exosomes, and as shown therein, it was confirmed that the contained exosomes did not affect changes in the transparency and color of the hyaluronic acid filler.

FIG. 3b shows confocal fluorescence microscope images of the hyaluronic acid fillers containing or not containing the exosomes, and as shown therein, it was confirmed that exosomes labeled with red fluorescence (Flamma Flour 675) were evenly distributed in the hyaluronic acid filler labeled with green fluorescence (Flamma Flour 496).

In addition, after the hydrogels containing or not containing the exosomes, injected by the above method, were freeze-dried, the insides of the hydrogel structures were observed through a scanning electron microscope, and the results are shown in FIG. 3c.

FIG. 3c shows scanning electron microscope images of the hyaluronic acid fillers containing or not containing the exosomes, and as shown therein, it was confirmed that the exosomes were evenly distributed in the hyaluronic acid filler having a mesh structure.

### 4-2. Analysis of Rheological Properties and Injection Force

A hyaluronic acid filler containing exosomes and a hyaluronic acid filler not containing exosomes were prepared according to the method of Example 3, and the storage moduli and loss moduli thereof at a frequency of 0.1 to 10 Hz at 25° C. were measured using a rheometer.

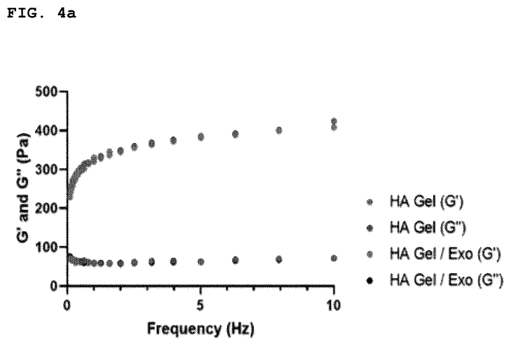

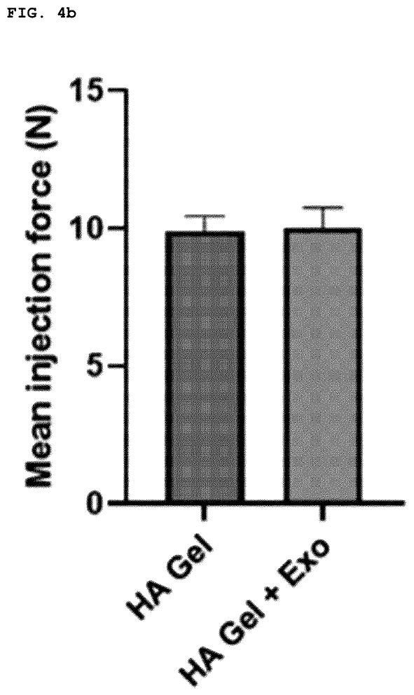

FIG. 4a shows the results of analyzing the rheological properties of the hyaluronic acid fillers containing or not containing the exosomes, and as shown therein, it was confirmed that the contained exosomes did not affect the storage modulus and loss modulus.

In addition, the hydrogel of each of the hyaluronic acid fillers containing or not containing the exosomes was placed in a 1 ml syringe, and the injection force thereof was measured using a universal testing machine.

FIG. 4b shows the results of analyzing the injection forces of the hyaluronic acid fillers containing or not containing the exosomes, and as shown therein, it was confirmed that the contained exosomes did not affect the injection force of the hyaluronic acid filler.

### Example 5. Evaluation of Anti-Aging Effect of Hyaluronic Acid Filler Using Animal Model

The effect of the hyaluronic acid filler containing human adipose stem cell-derived exosomes, prepared according to the method of Example 3, on collagen production in the skin layer, was evaluated using a mouse model.

Specifically, the hyaluronic acid filler containing the exosomes was injected into mice through intradermal injection so that the amount of the exosomes was 1×10⁷ exosomes/head, 5×10⁷ exosomes/head, or 1×10⁸ exosomes/head, and it was confirmed that the morphology of the hydrogel was maintained for 4 weeks. The skin layer was excised from the sacrificed mice, fixed in 4% formalin solution, embedded in paraffin, and sectioned into 4 μm tissue sections. The tissue sections were stained with H&E and Masson's trichrome and observed under an optical microscope to evaluate the side effects and collagen production effect of the hyaluronic acid filler.

In addition, the tissue sections were subjected to immunohistochemical staining using antibodies, which bind specifically to collagen I and III, and observed with a confocal optical microscope to determine the amounts of collagen I and III produced in the skin layer.

### Example 6. In Vitro Evaluation of Effect of Exosomes on Collagen Production by Activation of Anti-Inflammatory Macrophages

Cell experiments were conducted to evaluate the effect of the human adipose stem cell-derived exosomes according to the present invention on macrophages present in the skin layer to evaluate the effect of the exosomes on collagen production by improvement of the microenvironment. Normal macrophages (M0) were polarized into inflammatory macrophages (M1) by treatment with LPS (500 ng/mL) and IFN-γ (20 ng/mL) and into anti-inflammatory macrophages (M2) by treatment with IL-4 (20 ng/mL), and then treated for 48 hours with the human adipose stem cell-derived exosomes extracted in Example 1 to activate CD301b-expressing macrophages. Then, the activated macrophages were co-cultured with fibroblasts for 48 hours and subjected to cytotoxicity assay (CCK-8 assay) and Sircol collagen assay.

### Example 7. Evaluation of Effect of Exosome-Containing Hyaluronic Acid Filler on Improvement of Microenvironment in Skin Layer Using Animal Model

The effect of the hyaluronic acid filler containing human adipose stem cell-derived exosomes, prepared in Example 3, on collagen production by improvement of the microenvironment in the skin layer, was evaluated using a mouse animal model.

Specifically, the exosome-containing hyaluronic acid filler was injected into mice through intradermal injection so that the amount of the exosomes was 1×10⁷ exosomes/head, 5×10⁷ exosomes/head, or 1×10⁸ exosomes/head, and then the mice were sacrificed on day 7. The skin layer was excised from the sacrificed mice, fixed in 4% formalin solution, embedded in paraffin, and sectioned into 4 μm tissue sections. Thereafter, the tissue sections were subjected to immunohistochemical staining using anit-CD301b antibody and anti-ER-TR7 antibody, which bind specifically to CD301b and fibroblasts, respectively, and observed with a confocal optical microscope to evaluate the effect of the filler on improvement of the microenvironment in the skin layer.

### Experimental Examples

### Experimental Example 1. Evaluation of Exosome Release Behavior of Hyaluronic Acid Filler Depending on Content of BDDE

The hyaluronic acid filler synthesized to have each content of BDDE as described in Example 2 was mixed with the human adipose stem cell-derived, Flamma Flour 675-labeled exosomes extracted in Example 1, and then injected into animal models so that the amount of the exosomes was 1×10⁸ exosomes per animal. Then, differences in exosome release behavior between the fillers were examined by measuring the time-dependent intensity of the fluorescence labeled in the exosomes using a small animal in vivo imaging system (IVIS).

The biodistribution behavior of the fluorescently labeled exosomes contained in the hyaluronic acid filler was evaluated in real time using a small animal optical imaging system. Exosome-containing hyaluronic acid fillers were prepared by adjusting the content of BDDE to 6 levels (level 1:0, level 2: 10.85 mg, level 3: 54.23 mg, level 4: 108.46 mg, level 5: 162.69 mg, and level 6: 216.92 mg), and then the retention time of the fluorescently labeled exosomes in the skin layer was evaluated.

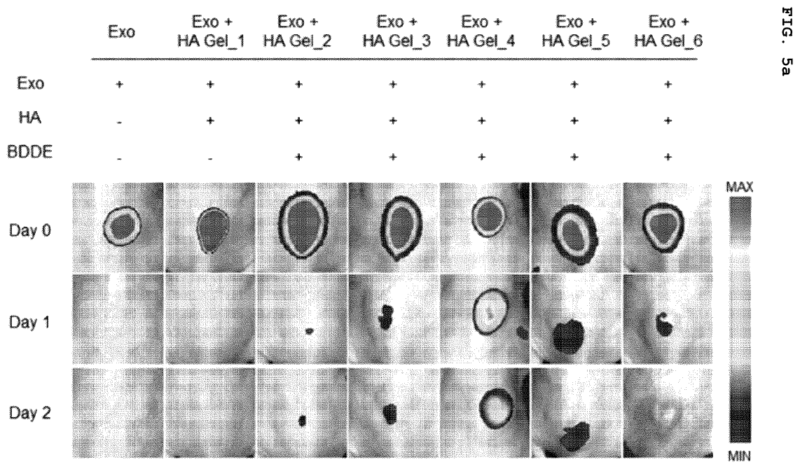

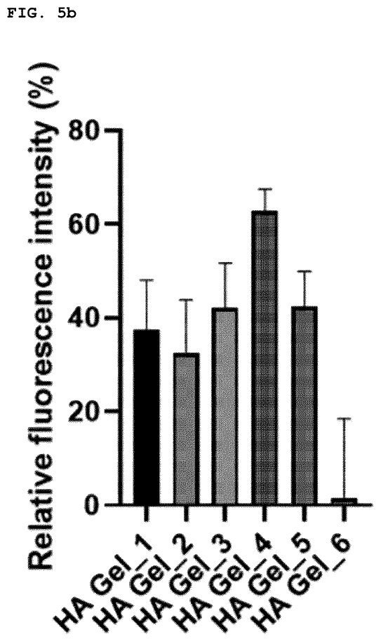

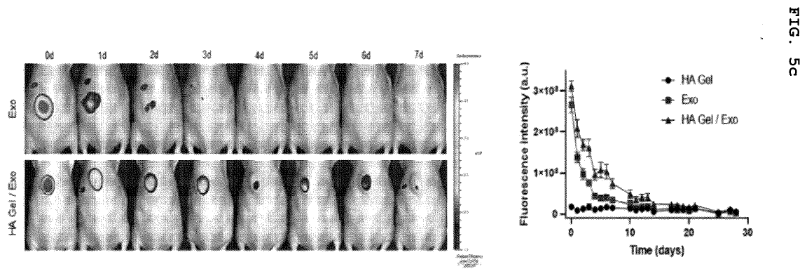

FIG. 5a shows the results of evaluating the biodistribution behavior of exosomes depending on the content of BDDE in the exosome-containing hyaluronic acid fillers, and as shown therein, it was confirmed that the fluorescently labeled exosomes present in the exosome-containing hyaluronic acid filler having a BDDE content of level 4 (108.46 mg) remained for the longest period of time.

FIG. 5b shows the results of evaluating the biodistribution behavior of exosomes 2 weeks after injection of the exosome-containing hyaluronic acid fillers, and as shown therein, it was confirmed that, compared to the initial injection, 63% of exosomes in the hyaluronic acid filler having a BDDE content of level 4 remained in the skin layer, but more than 50% of exosomes in the hyaluronic acid fillers having BDDE contents of the other levels disappeared outside of the skin layer.

In addition, FIG. 5c shows the results of evaluating the long-term biodistribution behavior of exosomes in the exosome-containing hyaluronic acid filler having a BDDE content of level 4, which is the optimal BDDE content identified in FIGS. 5a and 5b, and as shown therein, it was confirmed that exosomes contained in the hyaluronic acid filler remained in the skin layer for a significantly long time.

### Experimental Example 2. Evaluation of Collagen Production Effect of Injection of Hyaluronic Acid Filler in Mouse Model

The collagen production effect of injection of the exosome-containing hyaluronic acid filler was evaluated in a mouse model using the method of Example 5.

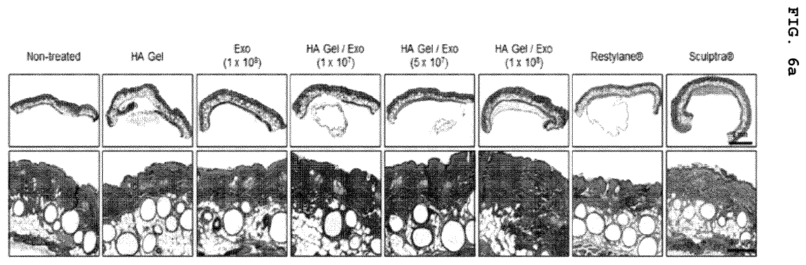

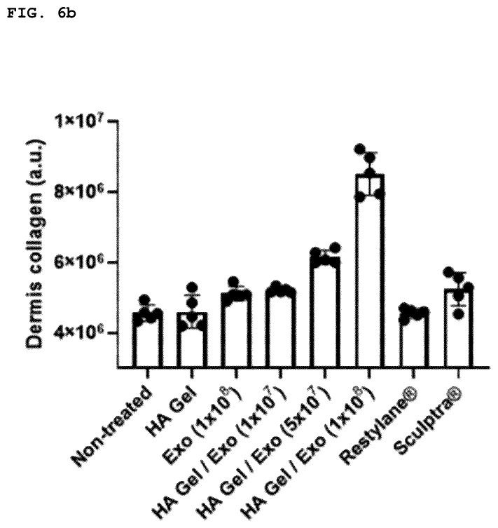

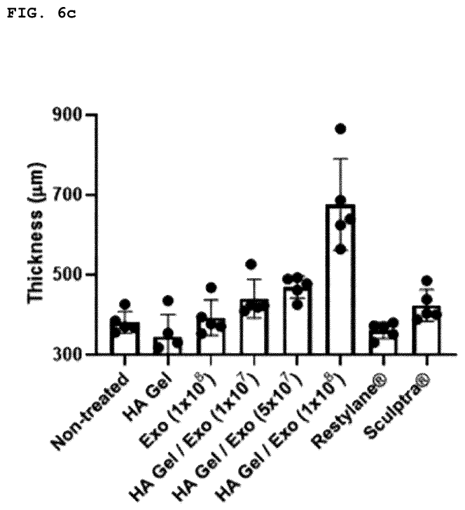

FIG. 6a shows the results of staining mouse skin layer tissue 24 weeks after injection of the exosome-containing hyaluronic acid filler. As shown therein, it was observed in histological analysis that, in the non-treated tissue, the tissue injected with the hyaluronic acid filler alone, and the tissue injected with the exosomes alone, no significant collagen production effect appeared, whereas, in the tissue injected with the exosome-containing hyaluronic acid filler, the dermal layer where collagen was distributed became thicker in an exosome concentration-dependent manner, suggesting that the exosome-containing hyaluronic acid filler exhibited an excellent collagen production effect. In addition, it was confirmed that Restylane®, which is the currently most widely used hyaluronic acid filler, and Sculptra®, which is a polymer-based filler, did not show histologically significant collagen production, suggesting that the exosome-containing hyaluronic acid filler according to the present invention exhibited an excellent collagen production effect.

FIG. 6b shows the results of quantitatively analyzing collagen in the dermal layer 24 weeks after injection of the exosome-containing hyaluronic acid filler. As shown therein, it was found that the collagen production effect of the exosome-containing hyaluronic acid filler was 1.9 times higher than that of the non-treated tissue, 1.7 times higher than that of injection of the exosomes alone, 1.9 times higher than that of injection of Restylane®, and 1.6 times higher than that of injection of Sculptra®.

FIG. 6c shows the results of quantitatively analyzing the thickness of the dermal layer in which collagen is distributed, 24 weeks after injection of the exosome-containing hyaluronic acid filler. As shown therein, it was found that the thickness of the dermal layer injected with the exosome-containing hyaluronic acid filler increased by 1.8 times compared to that of the non-treated tissue, 1.7 times compared to that of the dermal layer injected with the exosomes alone, 1.9 times compared to that of the dermal layer injected with Restylane®, and 1.6 times compared with that of the dermal layer injected with Sculptra®.

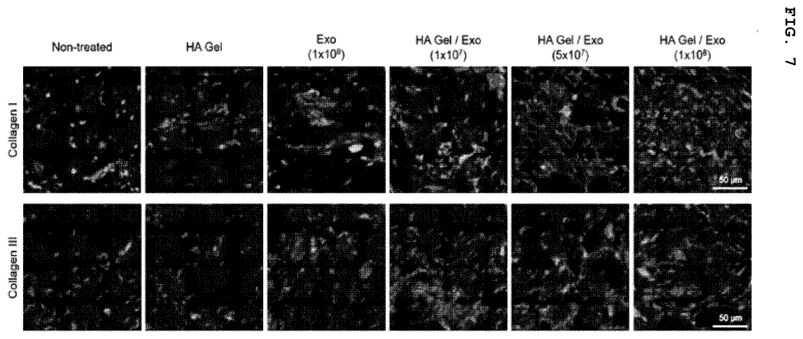

In addition, FIG. 7 shows the results of analyzing the production of collagen I and III in skin tissue after injection of the exosome-containing hyaluronic acid filler by immunohistochemical staining using a mouse model. As shown therein, 4 weeks after sample injection, the non-treated tissue, the tissue injected with the hyaluronic acid filler alone, and the tissue injected with the exosomes alone did not show significant production of collagen I and III (red fluorescence) in histological analysis, whereas injection of the exosome-containing hyaluronic acid filler showed the effect of producing high-density collagen I and III in an exosome concentration-dependent manner.

### Experimental Example 3. Evaluation of Effect of Exosomes on Collagen Production by Activation of Anti-Inflammatory Macrophages

The effect of human adipose stem cell-derived exosomes on collagen production by activation of anti-inflammatory macrophages was evaluated in vitro using the method of Example 6.

### 3-1. Analysis of Expression of Anti-Inflammatory Marker CD301b in Macrophages

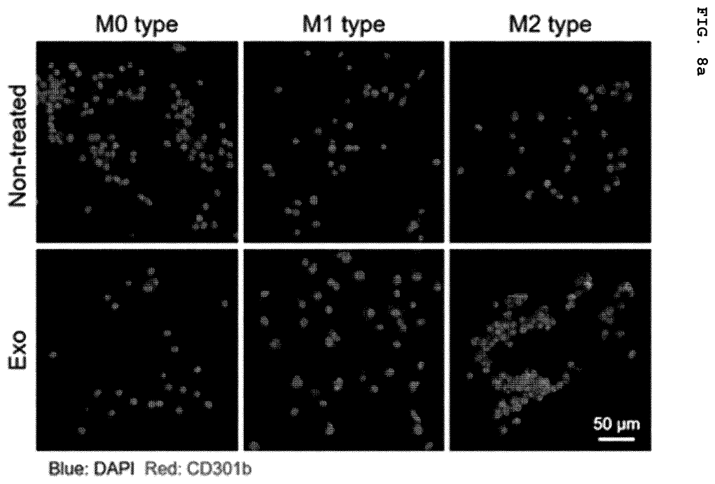

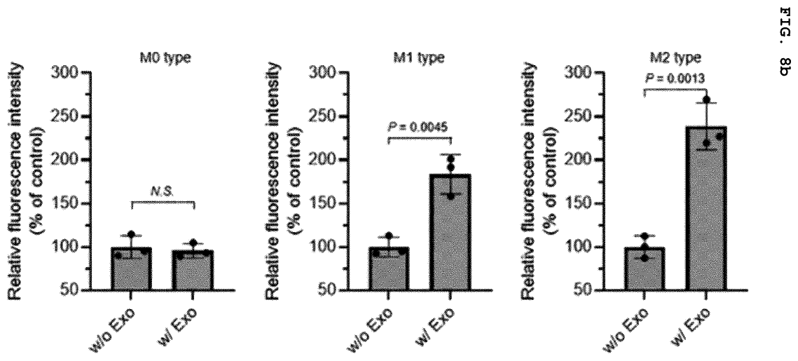

CD301b is a marker present in large amounts mainly in anti-inflammatory macrophages. FIG. 8a shows the results of evaluating the expression level of the anti-inflammatory marker CD301b in macrophages of various phenotypes (M0: normal macrophages, M1: inflammatory macrophages, and M2: anti-inflammatory macrophages), present in the skin layer and activated by the human adipose stem cell-derived exosomes according to one embodiment of the present invention (scale bar=50 μm). As shown therein, when treated with the exosomes, the expression level of CD301b was insignificant in normal macrophages (M0) and inflammatory macrophages (M1), whereas the expression level significantly increased in anti-inflammatory macrophages (M2). This suggests that the human adipose stem cell-derived exosomes had a significant effect on the expression of CD301b in macrophages involved in anti-inflammatory responses.

FIG. 8b shows the results of quantitatively analyzing CD301b expressed on the surface of macrophages, 48 hours after treating M0, M1, and M2 type macrophages with the exosomes. As shown therein, it was confirmed that, when macrophages of each phenotype were treated with the exosomes, the expression of CD301b in M0 was not substantially different from that in the macrophages not treated with the exosomes, and the expression of CD301b increased by 1.8 times in M1 and 2.4 times in M2.

### 3-2. Evaluation of Proliferation of Fibroblasts

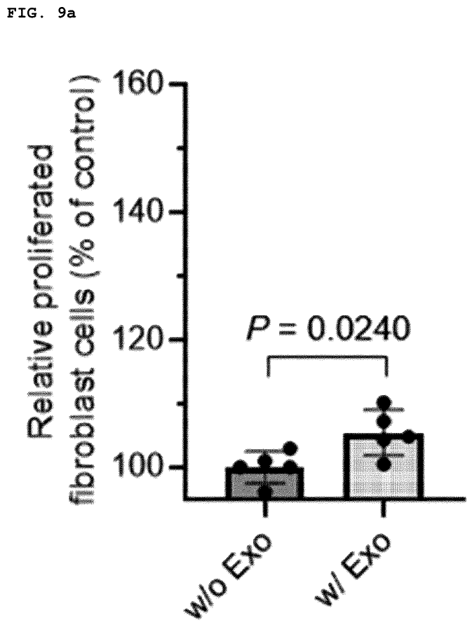

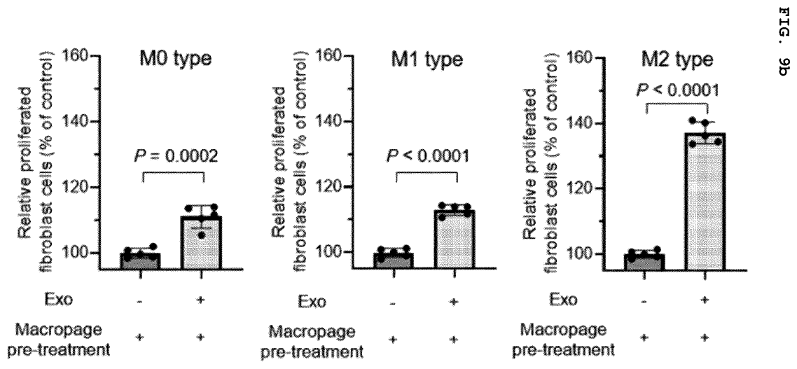

FIG. 9a shows the results of quantitatively analyzing the number of proliferated fibroblasts by cytotoxicity assay 48 hours after treatment with the exosomes to evaluate the effect of the exosomes on fibroblast proliferation. As shown therein, after 48 hours after treatment with the exosomes, the number of fibroblasts slightly increased by 1.05 times compared to that of fibroblasts not treated with the exosomes.

FIG. 9b shows the results of quantitatively analyzing the proliferation rate of fibroblasts by activated macrophages after co-culture of fibroblasts and the M0, M1, and M2 macrophages activated by treatment with the exosomes. As shown therein, it was confirmed that the number of fibroblasts co-cultured with the macrophages activated by the exosomes increased about 1.1 times when co-cultured with M0, about 1.1 times when co-cultured with M1, and about 1.4 times when co-cultured with M2, compared to when co-cultured with inactive macrophages not treated with the exosomes.

### 3-3. Evaluation of Collagen Synthesis Effect of Fibroblasts

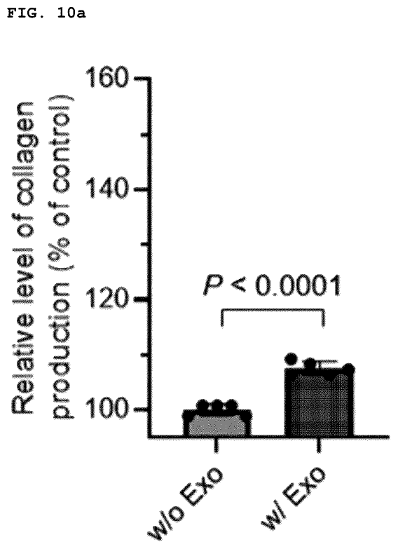

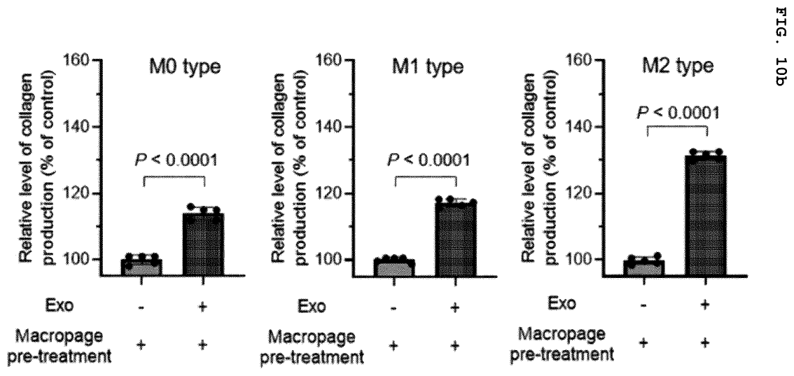

FIG. 10a shows the results of quantitatively analyzing collagen synthesized by fibroblasts 48 hours after treatment with the exosomes in order to evaluate the effect of the exosomes on collagen synthesis in fibroblasts. As shown therein, 48 hours after fibroblasts were treatment directly with the exosomes, the amount of collagen produced slight increased by 1.07 times compared to when fibroblasts were not treated with the exosomes.

FIG. 10b shows the results of quantitatively analyzing the collagen synthesis effect of fibroblasts induced by activated macrophages after co-culture of fibroblasts and the M0, M1, and M2 macrophages activated by pre-treatment with the exosomes. As shown therein, it was confirmed that collagen synthesis in the fibroblasts co-cultured with the macrophages activated by the exosomes increased by 1.14 times when co-cultured with M0, 1.17 times when co-cultured with M1, and 1.31 times when co-cultured with M2.

From the above results, it could be seen that the human adipose stem cell-derived exosomes according to the present invention further increased collagen synthesis in fibroblasts by activating anti-inflammatory macrophages in the skin layer.

### Experimental Example 4. Evaluation of Effect of Injection of Exosome-Containing Hyaluronic Acid Filler on Improvement of Microenvironment in Skin Layer

### 4-1. Analysis of Expression of Anti-Inflammatory Marker CD301b in Macrophages

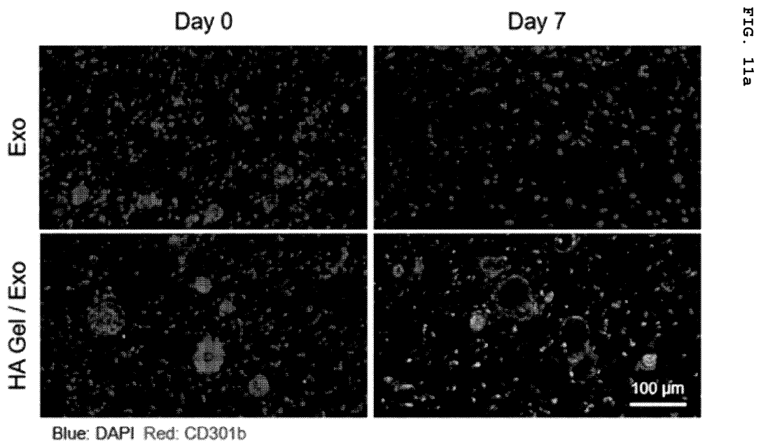

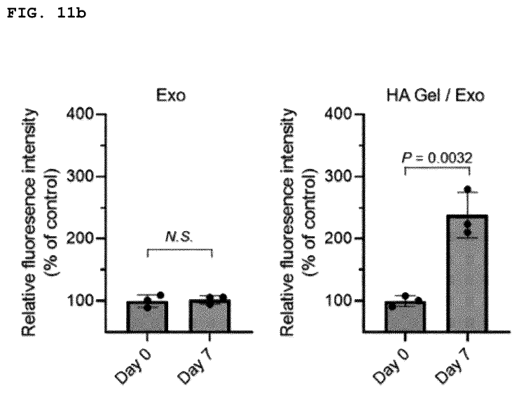

FIG. 11a shows the results of analyzing the expression of CD301b, an anti-inflammatory macrophage marker, in the skin layer after injecting the exosome-containing hyaluronic acid filler into a mouse model. As shown therein, it was confirmed that, 7 days after sample injection, the tissue injected with the exosomes alone did not show a significant increase in the expression of CD301b, whereas, when the exosome-containing hyaluronic acid filler was injected, the expression of CD301b in macrophages in the tissue significantly increased.

FIG. 11b shows the results of quantitatively analyzing the intensity of CD301b fluorescence (red fluorescence) in the images obtained with a confocal fluorescence microscope after immunohistochemical staining. As shown therein, it was confirmed that the CD301b expression level in the tissue day 7 after injection of the exosomes was 1.01 times higher compared to that on day 0, which was not significantly different, whereas, when the exosome-containing hyaluronic acid filler was injected, the expression level of CD301b in the tissue on day 7 increased by 2.38 times compared to that on day 0.

### 4-2. Evaluation of Proliferation of Fibroblasts

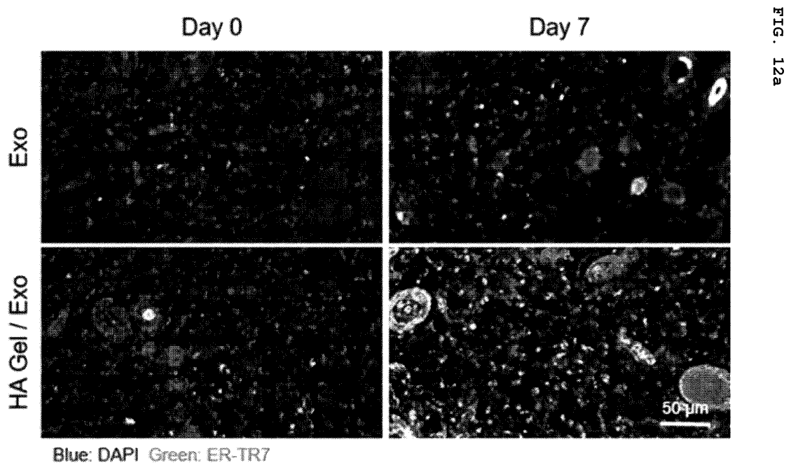

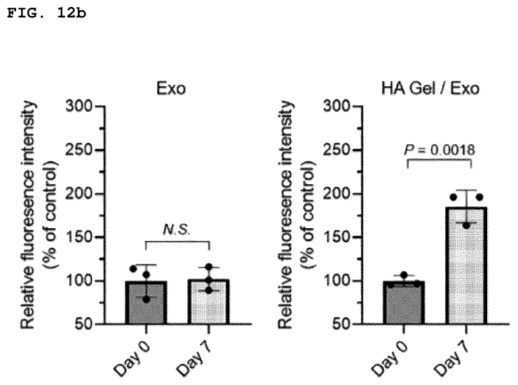

FIG. 12a shows the results of evaluating the proliferation of fibroblasts in the skin layer after injecting the exosome-containing hyaluronic acid filler into a mouse model. As shown therein, it was confirmed that, day 7 after sample injection, the tissue injected with the exosomes alone did not show a significant increase in the number of fibroblasts, whereas the number of fibroblasts significantly increased in the tissue injected with the exosome-containing hyaluronic acid filler.

FIG. 12b shows the results of quantitatively analyzing the number of fibroblasts (green fluorescence) in the images obtained with a confocal fluorescence microscope after immunohistochemical staining. As shown therein, it was confirmed that the number of fibroblasts in the tissue day 7 after injection of the exosomes was 1.02 times higher compared to that on day 0, which was not significantly different, whereas, when the exosome-containing hyaluronic acid filler was injected, the number of fibroblasts in the tissue day 7 increased by 1.86 times compared to that on day 0.

As described above, it was confirmed that the hyaluronic acid filler containing human adipose stem cell-derived exosomes according to the present invention had an anti-aging effect by exhibiting an excellent collagen production effect, and that the exosomes had a better effect when administered at 1×10⁸ exosomes/head compared to when administered at 1×10⁷ exosomes/head and 5×10⁷ exosomes/head.

The above description of the present invention is exemplary, and those of ordinary skill in the art will appreciate that the present invention can be easily modified into other specific forms without departing from the technical spirit or essential characteristics of the present invention. Therefore, it should be understood that the exemplary embodiments described above are exemplary in all aspects and are not restrictive.

1. A filler composition for reducing skin wrinkles, containing exosomes derived from stem cells, hyaluronic acid, and 1,4-butanediol diglycidyl ether (BDDE) as active ingredients.

2. The filler composition according to claim 1, wherein the stem cells are human adipose stem cells.

3. The filler composition according to claim 1, wherein the hyaluronic acid is crosslinked with BDDE to form a hydrogel.

4. The filler composition according to claim 1, wherein a dry weight ratio of the hyaluronic acid: BDDE is 1: 0.001 to 0.05.

5. The filler composition according to claim 1, wherein the filler composition activates anti-inflammatory macrophages.

6. The filler composition according to claim 5, wherein the filler composition increases expression of CD301b, an anti-inflammatory marker, in macrophages.

7. The filler composition according to claim 5, wherein the filler composition increases fibroblast proliferation and collagen production by activating anti-inflammatory macrophages.

8. A method for preparing a filler composition for reducing skin wrinkles, the method comprising steps of:
(a) extracting exosomes from stem cells;
(b) crosslinking hyaluronic acid by adding BDDE thereto;
(c) dialyzing the crosslinked hyaluronic acid using a dialysis membrane; and
(d) mixing the cross-linked hyaluronic acid after the dialyzing with the exosomes.

9. The method according to claim 8, wherein the stem cells are human adipose stem cells.

10. The method according to claim 8, wherein a dry weight ratio of the hyaluronic acid: BDDE in step (b) is 1:0.001 to 0.05.

11. The method according to claim 8, wherein the crosslinking in step (b) is performed at a temperature of 20° C. to 60° C. for 12 to 36 hours.

12. The method according to claim 8, wherein the dialyzing in step (c) is performed for 36 to 60 hours.

13. A method for reducing skin wrinkles, comprising a step of administering to a subject in need thereof a filler composition containing exosomes derived from stem cells, hyaluronic acid, and 1,4-butanediol diglycidyl ether (BDDE) as active ingredients.

14. Use of exosomes derived from stem cells, hyaluronic acid, and 1,4-butanediol diglycidyl ether (BDDE) for preparing a filler for reducing skin wrinkles.

15. Use of a filler composition containing exosomes derived from stem cells, hyaluronic acid, and 1,4-butanediol diglycidyl ether (BDDE) as active ingredients for reducing skin wrinkles.
